(19) United States
(12) Patent Application Publication
(10) Pub. No.: US 2008/0200957 A1
(43) Pub. Date: Aug. 21, 2008

(54) Pin for Anchorage of Articular Prosthesis, Articular Prosthesis Comprising Said Pin, Tibial Component and Articular Prosthesis for the Knee Comprising Said Tibial Component

(75) Inventors: Maurilio Marcacci, Bologna (IT); Daniele Pressato, Montegrotto Terme (IT); Sergio Di Fede, Bologna (IT); Claudio De Luca, Padova (IT); Anna Tampieri, Faenza (IT)

Correspondence Address: PEARNE & GORDON LLP, 1801 EAST 9TH STREET, SUITE 1200, CLEVELAND, OH, 44114-3108, US

(73) Assignee: FIN-CERAMICA FAENZA S.P.A., I-48018 FAENZA (RAVENNA) (IT)

(21) Appl. No.: 12/065,039

(22) PCT Filed: Sep. 4, 2006
(86) PCT No.: PCT/IT2006/000639

§ 371 Date: Apr. 23, 2008

(30) Foreign Application Priority Data
Sep. 6, 2005 (IT) MI2005A001634
Aug. 22, 2006 (IT) MI2006A001633

(51) Int. Cl. A61B 17/56

(52) U.S. Cl. 606/329

(57) ABSTRACT

A pin for the anchoring of articular prostheses includes a connecting portion (4) with a prosthesis (2) and an engagement portion (5) in a hole obtained within a bone. The engagement portion (5) is at least partly deformable in a radial direction and in a resilient way, in order to allow the instantaneous blocking of the pin (1) in the hole and the primary fixing of the prosthesis (2) on the bone, so as to al low an easy primary fixing of the prosthesis by simply introducing with a pressure the connecting portion in the hole obtained within the bone itself.

The present invention relates to an anchor pin of articular prostheses and an articular prosthesis having such pin.

The present invention further relates to a tibial component and a knee articular prosthesis including such tibial component.

The present invention is placed in the field of the orthopedic surgery and relates to a fixing system for mini-prostheses made, for example of titanium, chrome-cobalt alloy or other metal alloys or of ceramic materials, for different articular compartments.

The present invention finds an advantageous application in the ambit of the knee prosthesis, in particular in mono-compartment mini-prostheses intended for the knee joint (fixing to the femoral condyle and fixing to the tibial plate, fixing to the kneecap).

The knee joint, as any other articular compartment, is coated by a hyaline cartilage coating which, due to the wear, is exposed to a degenerative pathology (arthrosis) which can concern, in the most serious morbid forms thereof, the femoral and/or tibial and/or of the kneecap sub-cartilage bone. Adjacent articular surfaces, in such pathological conditions, can cause pain, rigidity, loss of articular functionality.

Generally, the morbid forms of the articular cartilage in their most advanced stadium involve the need of a surgical operation aimed to restore the lost articular surface, through the positioning, referring to the knee, between the arthro-prostheses, of a mono-compartment prosthesis, if the affection is restricted to a single cartilage compartment, so as to restore the normal sliding of the femoral articular portion on the tibial one.

Therefore, the prosthesis includes a condyle-femoral component and a tibial component, sometimes defined by the term tibial plate since, once installed, it functionally replaces the bone tibial plate.

As it is known, each of the condyle and tibial components consists of a plate having one or more anchor pins or bars intended for the engagement in respective seats previously obtained by incision in the femoral bone and the tibial one. The pins must be immediately blocked in the respective holes, in order to ensure the primary stability of the prosthesis, and should subsequently allow the osteo-integration of the prosthesis with the adjacent bone, if cement is not used for the fixing.

When the installation is completed, the condyle-femoral component abuts and slides on the tibial component, which, for this purpose, shows a coupling surface with the condyle-femoral component made of a material showing a low friction resistance, for example very high density polyethylene.

For the purpose of ensuring the primary stability, pins which are blocked in the respective holes by means of screws which pass through the bone and are transversally engaged in the pin itself are known. Such structure does not allow an immediate and simple blocking of the prosthesis and further requires the execution of additional holes in the bone for the housing of the screws.

Alternatively, the pin is cemented in the hole through a proper bone cement (in this case the stability is not of an osteo-integrative type).

Moreover, the pins and the bars of a known type are solid bodies without seats capable of containing the parent cells appointed to the synthesis of bone matrix (osteoblasts) deriving from the marrow and the cells appointed to the bone remodelling (osteoclasts). Furthermore, a synthetic bone substitute suitable for promoting the osteo-integration results of a difficult application, as available empty volumes are not present. Accordingly, in the pins of a known type, the element on which one has to work and promote an optimal osteo-integration is the only outer surface, which can be modified, if necessary, through blasting or chemical erosion in order to obtain a certain roughness which allows the anchoring of the cells assigned to the synthesis of a new bone.

Furthermore, if necessary, also to the surface of the plate of the tibial component which remains into contact with the bone a predetermined roughness is imparted, for the purpose of obtaining what already specified above.

In this case, the technical task placed at the base of the present invention is to plan an anchor pin of articular prostheses capable of substantially obviating to the mentioned limits.

Mainly, an aim of the present invention is to plan an anchor pin of articular prostheses which ensures a safe and exact primary fixing.

In particular, an aim of the present invention is to propose a pin which can be easily and quickly introduced and blocked in the hole obtained in the bone.

In the ambit of said technical task an important aim of the invention is then to plan an anchor pin of articular prostheses which ensures the precise positioning of the prosthesis and the stability of the same.

Furthermore, an aim of the present invention is the invention of a pin which ensures a complete osteo-integration, by promoting the migration of the bone cells and the growth of the bone neoplasia.

A further aim of the invention is to plan an articular prosthesis which can be easily and stably fixed on the bone.

In this situation, the technical task placed at the base of the present invention is also to plan a tibial component capable of substantially obviating to the mentioned limits.

Mainly, an aim of the present invention is to plan a tibial component which ensures a safe and exact primary fixing.

In particular, an aim of the present invention is to propose a tibial component which can be easily and quickly introduced and blocked in a seat obtained in the bone of the tibial plate.

In the ambit of said technical task an important aim of the invention is then to plan a tibial component which ensures the precise positioning of the prosthesis and the stability of the same.

Furthermore, an aim of the present invention is the invention of a tibial component which ensures a complete osteo-integration, by promoting the migration of the bone cells and the growth of the new bone.

Finally, an aim of the invention is to plan a knee articular prosthesis including such tibial component which can be easily and stably fixed on the bone.

The stated technical task and the specified aims are substantially attained by a pin for articular prostheses having the features reported in one or more of the claims 1 to 11 and by an articular prosthesis according to one or more of the claims 12 to 20.

The stated technical task and the specified aims are further substantially attained by a tibial component having the features reported in one or more of the claims 21 to 55 and by a knee articular prosthesis according to the claims 56 and 57.

It is now reported, by way of not limitative example, the description of two preferred, but not exclusive, embodiments of a pin for articular prostheses according to the invention, and a preferred, but not exclusive, embodiment of a tibial component according to the invention, shown in the appended drawings, in which:

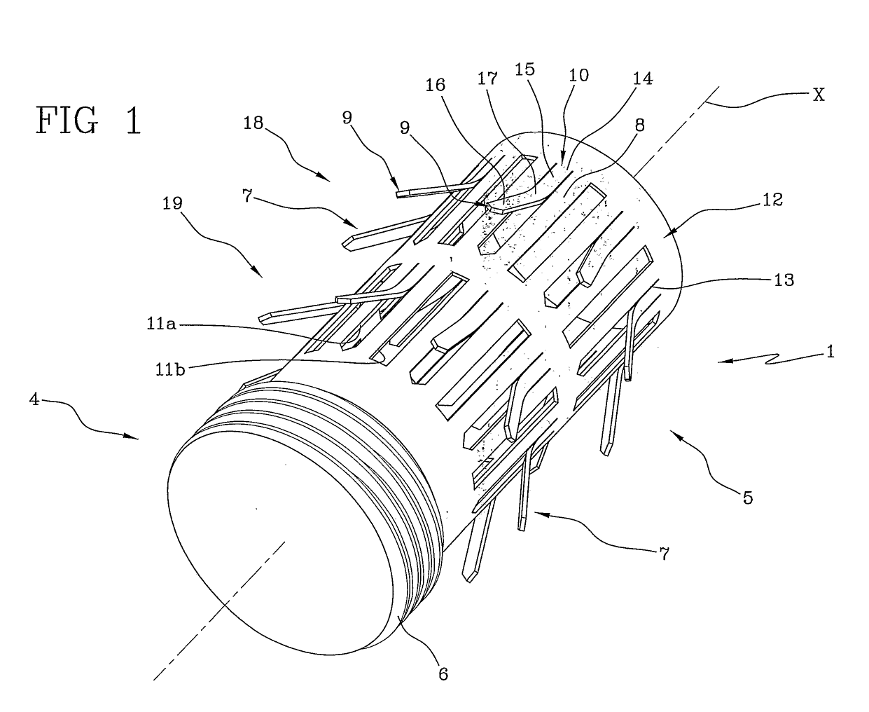

FIG. 1 shows a perspective view of a pin for articular prostheses according to the present invention, according to a first embodiment and in a first working configuration;

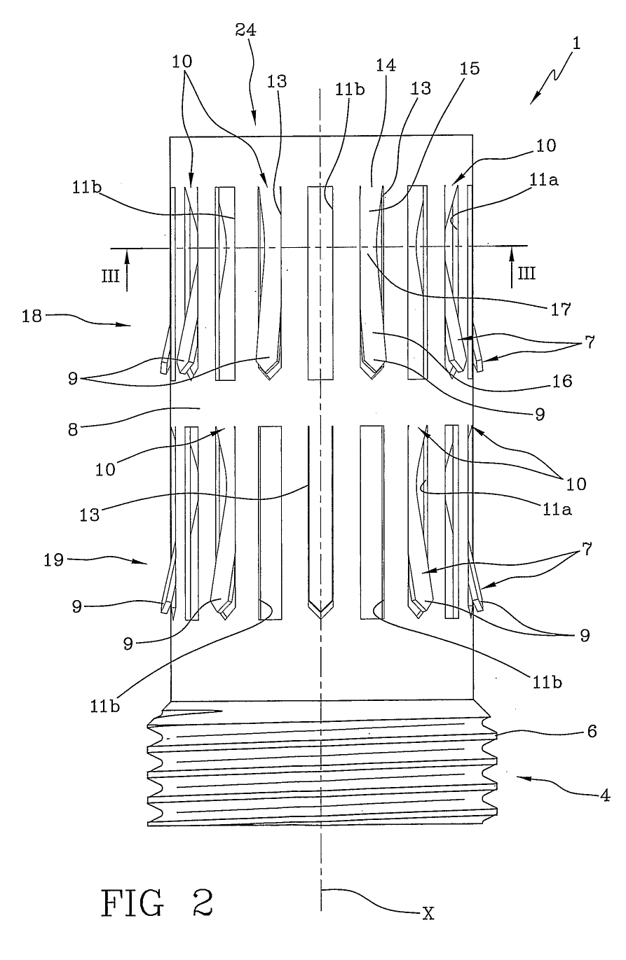

FIG. 2 shows a side elevation view of the pin of FIG. 1 in a second working configuration;

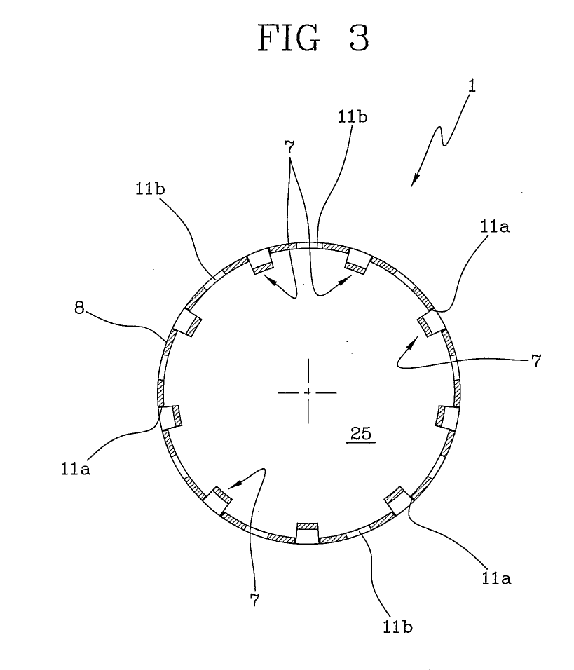

FIG. 3 shows a section of the pin according to the line III-III of FIG. 2;

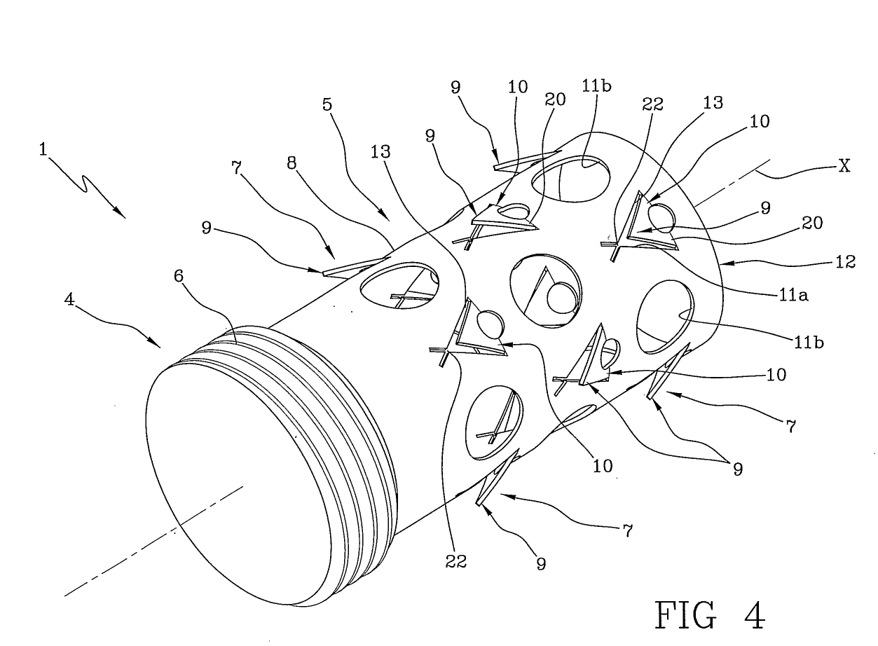

FIG. 4 shows in a perspective view a second embodiment of the pin of FIG. 1 in the first working configuration;

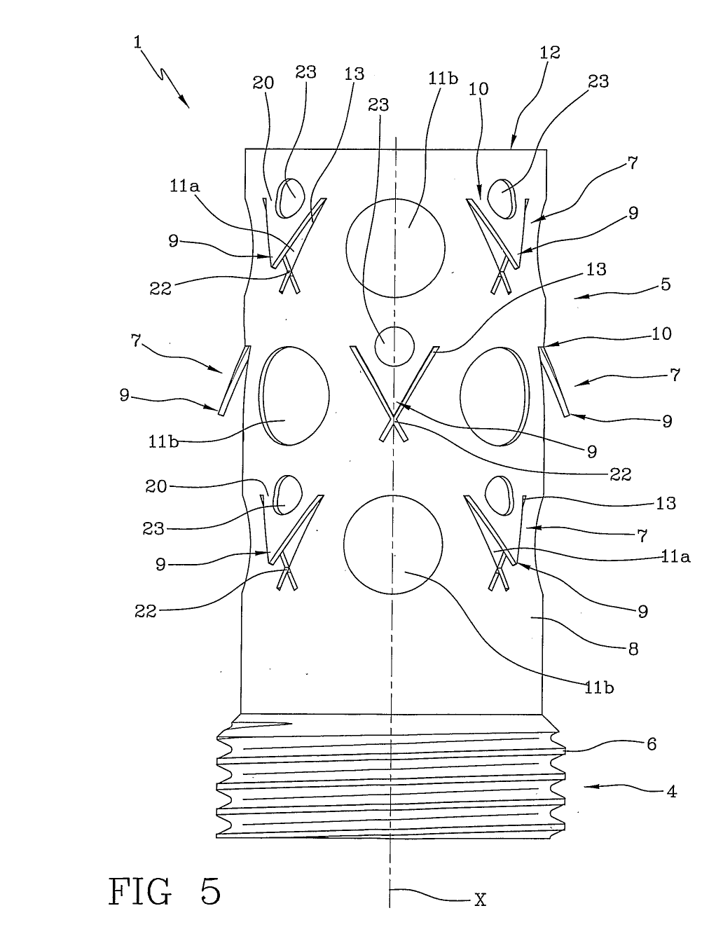

FIG. 5 shows a side elevation view of the pin of FIG. 4 in the second working configuration;

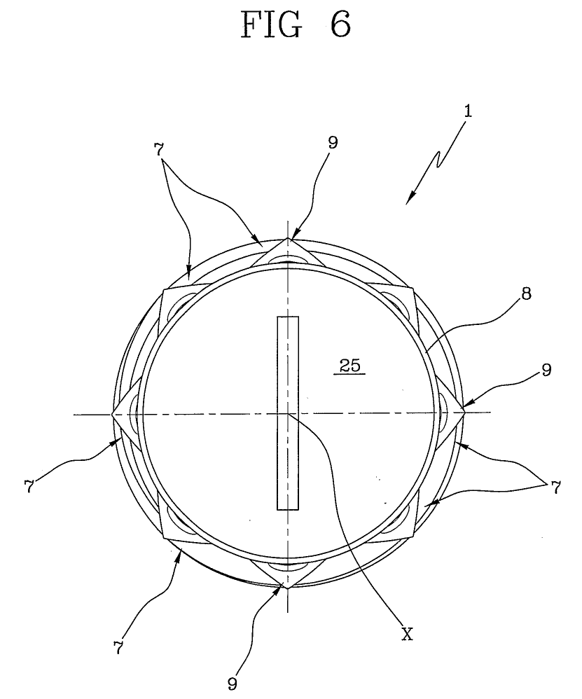

FIG. 6 shows a top view of the pin of FIG. 4; and

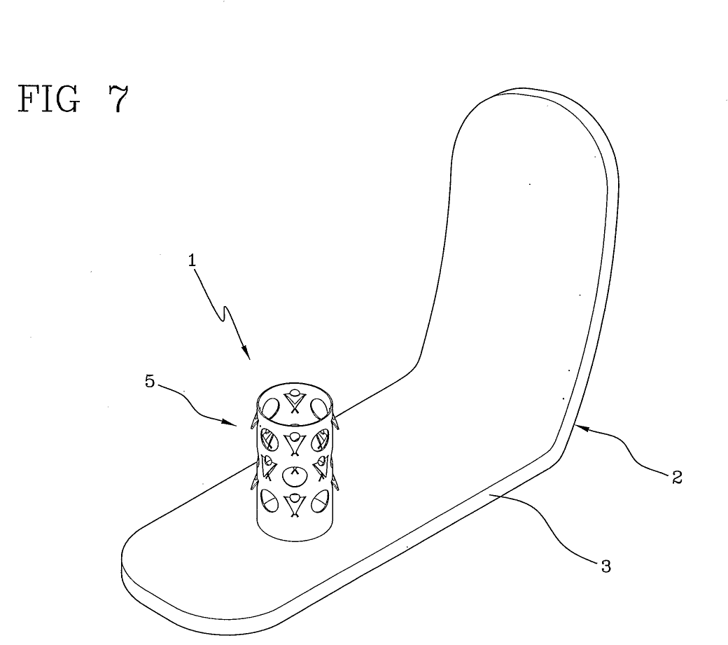

FIG. 7 diagrammatically shows an articular prosthesis equipped with the pin of FIGS. 1-6;

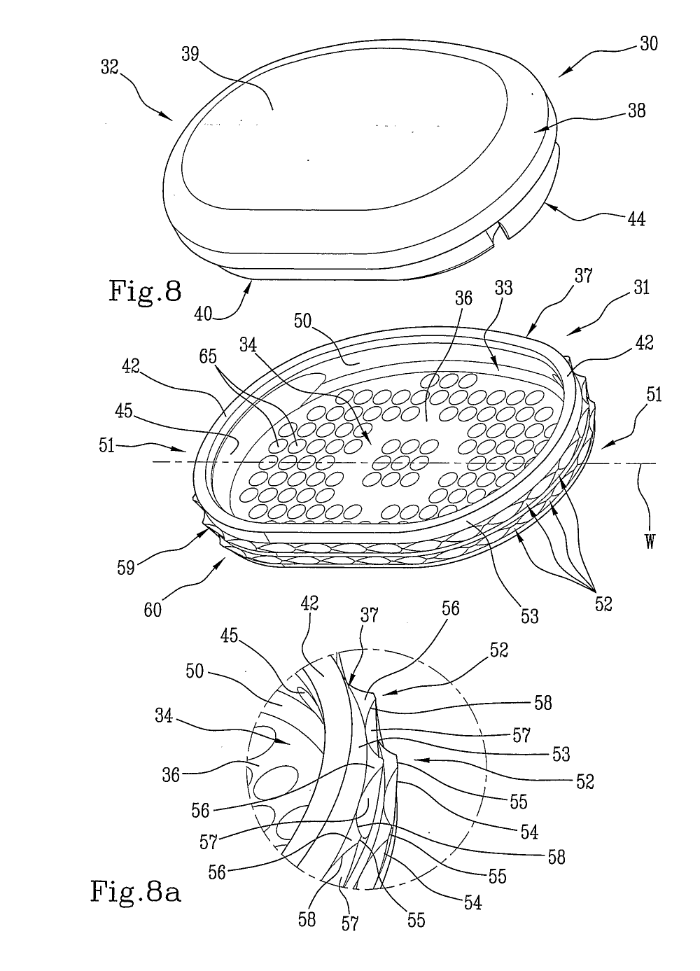

FIG. 8 shows a perspective and exploded view of a tibial component according to the present invention;

FIG. 8a is an enlargement of a portion of one of the elements of the tibial component of FIG. 8;

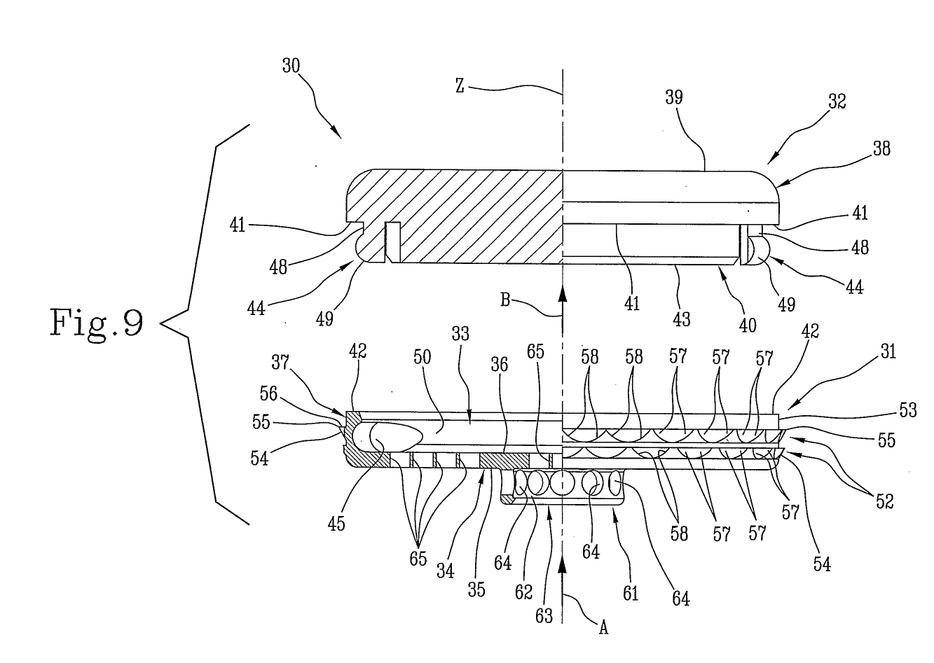

FIG. 9 shows a partly sectional and exploded side elevation view of the tibial component of FIG. 8;

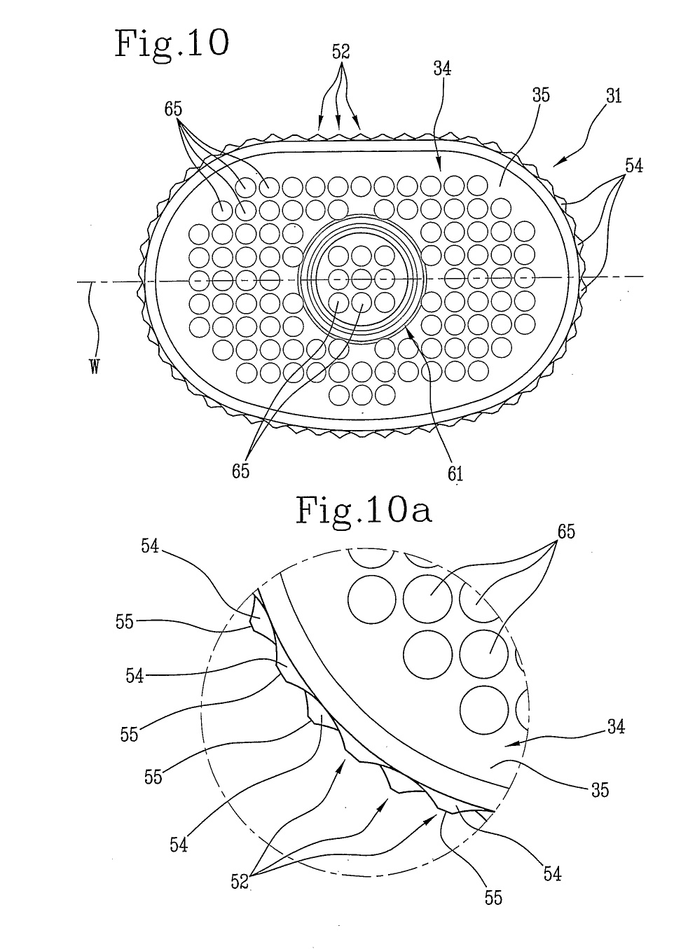

FIG. 10 shows a plan view according to the arrow “A” of FIG. 9 of an element of the tibial component of FIGS. 8 and 9;

FIG. 10a is an enlargement of a portion of the element of FIG. 10; and

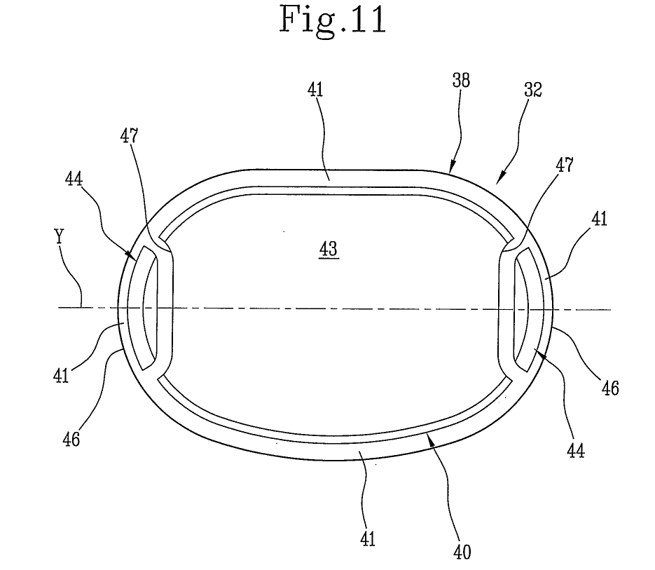

FIG. 11 shows a plan view according to the arrow “B” of FIG. 9 of an element of the tibial component of FIGS. 8 and 9;

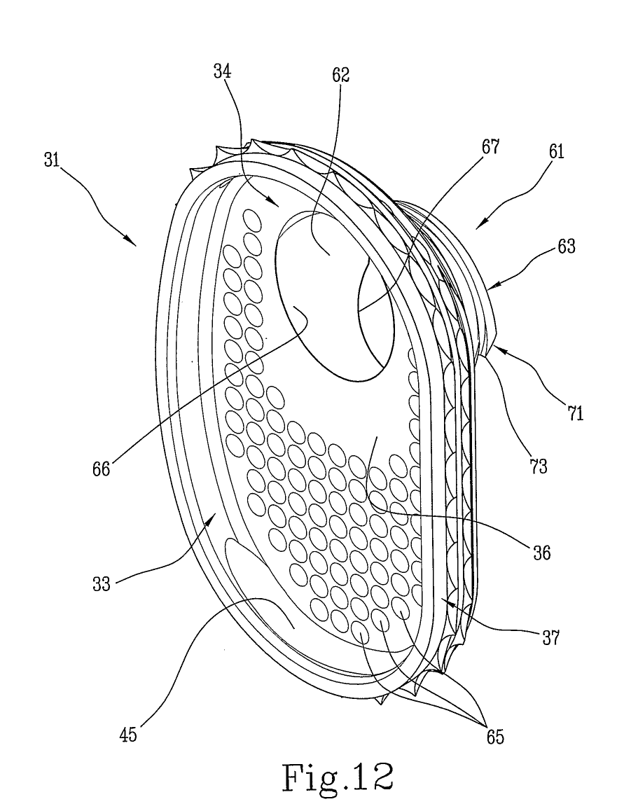

FIG. 12 shows in a perspective view an execution variant of an element of the tibial component of FIG. 8;

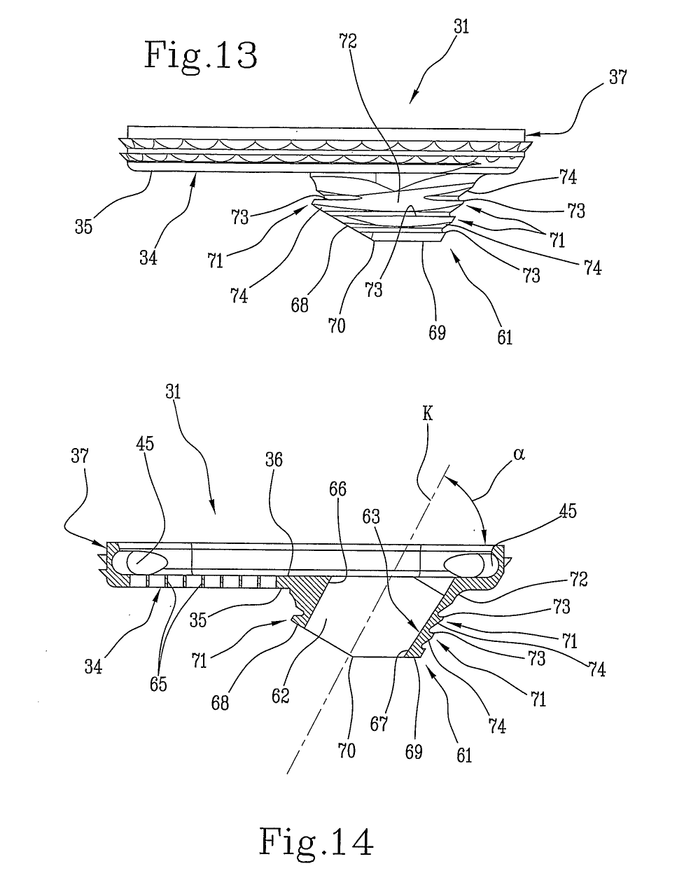

FIG. 13 shows a side elevation view of the element of FIG. 12;

FIG. 14 is a section of the side elevation view of FIG. 13; and

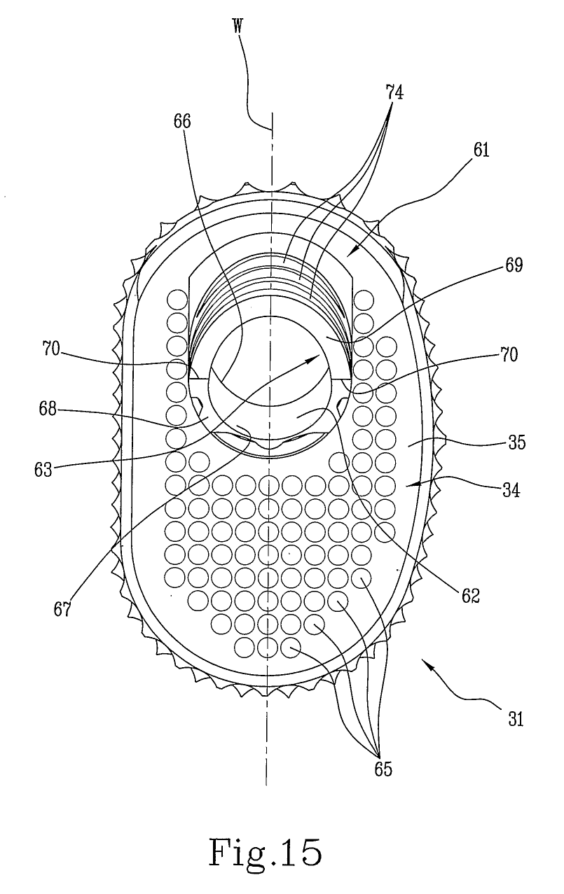

FIG. 15 shows the element of FIG. 12 in a plan view according to the arrow “C” of FIG. 13;

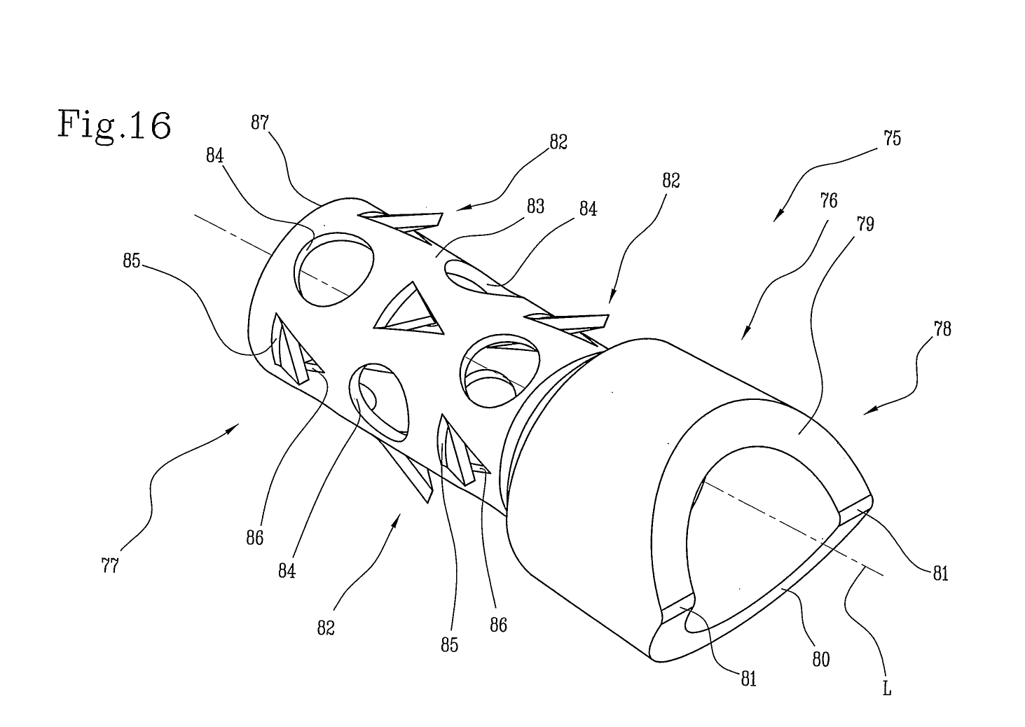

FIG. 16 shows in a perspective view a further element of the tibial component of FIG. 8;

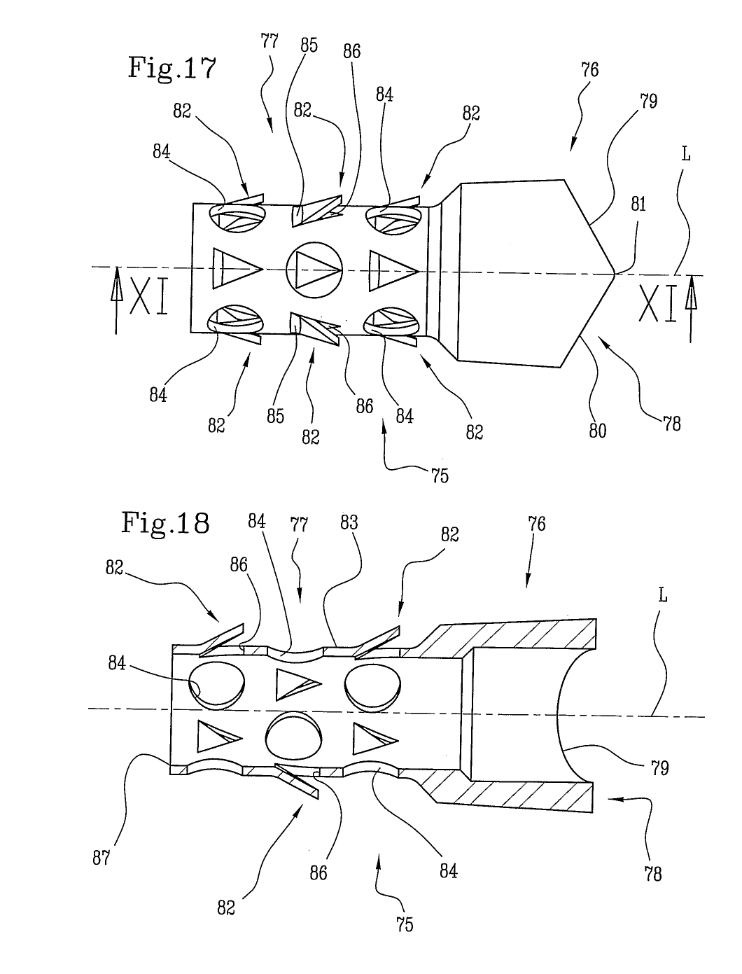

FIG. 17 is a side view of the additional element of FIG. 16;

FIG. 18 is a longitudinal section according to the line XI-XI of FIG. 17 of the additional element of FIG. 16.

Referring to the mentioned figures, a pin for articular prostheses according to the invention is generally shown by numeral 1 and a tibial component for knee articular prostheses according to the invention is generally shown by numeral 30.

In the non-limiting example shown in FIG. 7, the pin 1 is associated with a knee prosthesis 2 defined by a half-moon-shaped plate 3 and intended for being associated with the femoral condyle. The pin 1 could how-ever belong to the tibial component 30, sometimes defined by the term tibial plate, or other kinds of prostheses intended for any articular compartment, not shown.

The pin 1 is extending along an own rectilinear longitudinal axis “X” and includes a connecting portion 4 intended for being associated with the prosthesis 2 and an engagement portion 5 suitable for being introduced in a hole obtained in the bone.

In the preferred and shown embodiments, both the connecting portion 4 and the engagement one 5 of the pin 1 show a circular cross section and the connecting portion 4 is equipped with an outer threading 6 which can be engaged in a threaded hole, not shown, obtained in the plate 3.

According to additional embodiments, not shown but falling within the ambit of the present invention, the engagement portion 5 takes different shapes, for example with a polygonal section, while the connecting portion 4 is joined to the plate 3 with different techniques, for example by welding.

Furthermore, if the pin 1 is made of the same material of the plate 3, such as the titanium, the whole prosthesis 2 can be obtained in one piece, for example by molding or removal of material from a single metal block.

Advantageously, the engagement portion 5 of the pin 1 is at least partly deformable in a radial direction and in a resilient way, in order to allow the instantaneous blocking of the pin 1 in the hole and the primary fixing of the prosthesis 2 on the bone. The engagement portion 5 preferably includes a plurality of resilient elements 7 developing away from an own side outer surface 8 and which, subjected to a compression, are movable along a radial direction with respect to the axis “X” of the pin 1, between a resting configuration and a working configuration.

In the resting configuration, free ends 9 of the resilient elements 7 lie spaced from the side surface 8 of the pin 1. When the pin 1 is introduced in the hole, the internal wall of the same hole causes the deformation of the resilient elements 7 towards the axis “X” of the pin 1. Once the engagement portion 5 has been inserted, the elasticity of the mentioned elements 7 pushes the free ends 9 of the same against the internal wall of the hole, holding the pin 1 within the bone.

More particularly, according to what shown in the appended figures, the resilient elements 7 of the engagement portion 5 are defined by a plurality of resilient wings 7 which develop themselves from the outer surface 8.

Preferably, each wing 7 is joined to the engagement portion 5 in correspondence with an own restrained end 10 opposite to the free end 9 and mostly extending along a direction parallel to the longitudinal axis “X” of the pin 1.

Advantageously, furthermore, the engagement portion 5 is at least partly hollow and equipped with openings 11a, 11b so as to allow the housing of osteoinductive and osteoconductive material, such as the hydroxylapatite, and the passage of body fluids and medullar blood intended for forming the new bone material around to and within the pin 1.

In a first embodiment shown in FIGS. 1, 2 and 3, the engagement portion 5 is defined by a cylindrical wall 12 with a circular section which shows a plurality of notches 13. Each of the notches 13 delimits a wing 7 and defines a respective main opening 11a. The shown pin 1 presents a total length of 14 mm while the axial length of the connecting portion 5 is of 11.5 mm. The internal diameter of the connecting portion 5 is of 5.8 mm and the outer one is 6 mm.

Each main opening 11a has a rectangular shape with an own main side, 4 mm long, parallel to the longitudinal axis “X” of the pin 1. Therefore, each wing 7 has a substantially rectangular shape, is mainly developing along the direction parallel to the longitudinal axis X” and shows a lower side 14 joined to the cylindrical wall 8. The free end 9 of each wing 7 is faced towards the connecting portion 4 and is pointed, so as to ensure a more effective interference with the inner wall of the hole obtained within the bone.

Each wing 7 is further curved and shows a concavity faced towards the exterior of the pin 1. In particular, according to what shown in FIGS. 1, 2 and 3, the wing 7 includes a first part joined to the cylindrical wall 12 and a second part 16 which finishes with the pointed free end 9 and is separated from the first one by a bending 17.

In the resting configuration shown in FIG. 1, the first part 15 substantially lies in the wall 12 and the second part 16, due to the bending 17, is developed away from the wall 12 itself and is faced towards the connecting portion 4.

In the working configuration, shown in FIGS. 2 and 3, the free end 9 of the second part 16 is pushed towards the wall 12 and the wing 7 partly returns, particularly in correspondence with the bending 17, within the engagement portion 5. The first part 15 is elastically bended within said engagement portion 5 and ensures the radial thrust of the wing 7 against the wall of the hole in which the pin 1 is housed.

The pin 1 shown in FIGS. 1, 2 and 3 shows a first series 18 of wings 7 flanked to each other and circumferentially arranged around the longitudinal axis “X”. In particular, the wings 7 of the first series are nine and are angularly spaced of 40° from each other. Amongst each wing 7 and the following is also present an auxiliary opening 11b with a rectangular shape and dimensions substantially equal to those of the main openings 11a associated with the wings 7. The auxiliary openings 11b shown are nine and are angularly spaced of 40° from each other and of 20° from the adjacent wings 7.

The pin 1 further shows a second series 19 of wings 7 associated with respective auxiliary openings 11b axially spaced from the first series 18 of about 1 mm. The second series 19 of wings 7 is further angularly staggered with respect to the first one 18, such that each wing 7 of the first series 18 is aligned with an auxiliary opening 11b of the second series 19 along a generatrix of the engagement portion 5.

Finally, each main 11a and auxiliary 11b opening delimits, in a cross section visible in FIG. 3, a circle arc and subtends an arc of about 10°.

In a second embodiment, shown in FIGS. 4, 5 and 6, each wing 7 shows a triangular shape with a side 20 lying on a circumference directrix of the cylindrical part 12 and joined to the cylindrical wall 12 itself and a free vertex 21 faced towards the connecting portion 4. According to what shown, the triangle is isosceles with the junction side 20 which preferably measures 1.85 mm and the remaining sides of about 2 mm. Each wing 7 is obtained through a pair of rectilinear notches 13 which intersect in a crossing point 22 and define a main triangular opening 11a. In correspondence with the side 20 joined to the wall 12 an additional opening 23, whose function is to decrease the junction stiffness of the wing 7 with the wall 12, is further obtained. Preferably, such opening 23 is circular and shows a diameter of 0.8 mm.

The second embodiment shows three series of wings 7, each series is axially and angularly staggered from the adjacent series and includes four wings 7 angularly equispaced and the same amount of circular auxiliary openings 11b preferably having a diameter equal to 2 mm.

In both the embodiments shown, an end 24 of the engagement portion 5 opposite to the connecting portion 4 is open so as to allow the housing of a small cylinder of said osteoinductive mineral material, suitable for promoting the migration of the cells responsible for the osteo-integration, in the cylindrical recess 25 delimited from the wall 12.

The pin 1 carried out according to the invention attains important advantages.

In fact, the presence of the resilient wings or, more generally, the radial resilient deformability of the pin allows an easy primary fixing of the prosthesis on the bone, by a simply pressure fitting of the connecting portion in the hole obtained in the bone itself. Such advantage means a remarkable simplification of the surgical procedure, as it does not need the positioning in the bone housing of multiple elements, such as the screws, which represent the traditional fixing system.

Furthermore, the use of one or more pins according to the invention prevents any longitudinal and axial movements of the prosthetic structure in its general configuration immediately after the installation.

The hollow structure of the connecting portion and the presence of openings in the cylindrical wall allow the migration of cell components, sproutings of autologous bone and nutriment fluids rich in growth factors within the cylindrical empty structure of the anchor pin. All these biological components represent a determining factor for the bone neoplasia process.

Finally, the filling of the empty space of the anchor pin with a small cylinder of biomimetic bone substitute based on hydroxylapatite with a high porosity, simply placed by a “press-fit” represents a factor promoting the bone neoplasia process and accelerates, de facto, the osteo-integration within the seat obtained in the sub-cartilaginous bone. The scaffold will be occupied by a cell population which will undergo, thanks to the biomimetic properties of the same, a differentiation in an osteoblast sense with a consequent synthesis of bone matrix and promoting, thanks to it microporous network, the new angiogenesis.

In the non-limiting example shown in FIGS. 8 to 18, the tibial component 30 includes an anchor plate 31, which is restrained to the bone tibial plate of a patient, and an insert 32 made of a material with a low friction coefficient and highly resistant to the compression, preferably in very high density polyethylene.

Alternatively, the insert 32 can be made of other wear resistant polymers or in a ceramic material.

The plate 31 shows a seat 33 in which the insert 31 is housed, whose function is to replace the degenerated articular cartilage of the bone tibial plate.

In detail, the anchor plate 31 includes a base wall 34 which has a plan ovoidal or, more precisely, “bean-shaped” form (FIG. 10) which is plan developed mainly along a predefined direction “W”. The base wall 34 further presents a first face 35 and a second face 36 opposite and preferably plane therebetween.

A side wall 37 is developed along the peripheral edge of the base wall 34 and extends perpendicularly relative to the mentioned base wall 34, away from the second face 36, so as to delimit, together with the second face 36, the said seat 33 for the insert 31.

Preferably, the height of the side wall 37, or the extension of the same along an axial direction “Z” perpendicular to the surfaces 35, 36 of the plate 34, is included between 0.1 and 0.2 times the greater plan dimension of the plate 34.

The insert 32 shows a plan shape similar to that of the anchor plate 31 and includes a first portion 38 which has a working surface 39 intended for coming into contact with the condyle-femoral component. In the shown embodiment, such surface 39 is plane but in additional embodiments, not shown, it could take different conformations, depending on the requirements.

The insert 32 further shows a second portion 40 with reduced plan dimensions with respect to the first portion 38 and such to allow the introduction in the seat 33 of the anchor plate 31.

Between the first 38 and the second portions 40 a step delimiting an annular shoulder surface 41 (FIG. 9), lying on the first portion 38, opposite to the working surface 39 and surrounding the said second portion 40, is defined.

When the insert 32 is mounted in the seat 33, the second portion 40 is housed in such seat 33 and the annular shoulder surface 41 abuts against a final edge 42 of the side wall 37 of the anchor plate 31. A face 43 of the second portion 40 abuts against the second face 36.

Advantageously, the insert 32 is snap-mounted in the seat 33. For this purpose, the insert 32 includes at least a couple of elastically deformable teeth 44 and which can be snap-placed in respective housings 45 obtained in the seat 33.

In the preferred and shown embodiment, the insert 32 presents two teeth 44 placed in correspondence with opposite ends 46 of the said insert 32 lying along a predefined direction “Y” with a prevailing development (FIG. 11).

Each of the teeth 44 is developed away from the first portion 38 of the insert 32, lies flanked to the second portion 40 and sufficiently spaced from such second portion 40 to allow the bending of the tooth 44 itself. For this purpose, the tooth 44 is arranged in a recess 47 obtained in the second portion 40 (FIG. 11).

Each tooth 44 includes a part with a reduced section 48, in which proximity the bending occurs, joined to the first portion 38 and a distal end 49 having a counter-shaped thickening to the respective housing 45 (FIG. 9).

Within the seat 33, two housings 45 are obtained, defined by recesses carried out on an internal surface 50 of the side wall 37, in correspondence with opposite ends 51 of the plate 31 lying down along the said predefined direction “W”. The distal end 49 of each of the teeth 44 shows a curved plan shape which follows the plan profile of the recess 45.

The anchor plate 31 further includes a plurality of projections 52 arranged on an own peripheral side surface 53, which coincides with the outer surface of the side wall 37 and is faced from an opposite part relative to the seat 33.

The projections 52 allow the instantaneous blocking of the tibial component 30 in a housing obtained in the bone tibial plate of the patient and the primary fixing of such tibial component 30 on such tibial plate.

The projections 52 further show such a structure to allow an easy introduction in the bone and to simultaneously ensure the immediate blocking.

For this purpose, preferably, each projection 52 shows a surface 54 inclined with respect to the peripheral side surface 53, which inclined surface 54 is developed away from the peripheral side surface 53 starting from a zone of the tibial component 30 opposite to the mounting seat 33 of the insert 32 towards said seat 33.

In other words, the inclined surface 54 moves away from the outer surface 53 of the side wall 37 starting from an own proximal end close to the base wall 34 towards an own distal end closer to the final edge 42 of the side wall 37 (FIG. 9). Therefore, the projections 52 generally diverge approaching to the final edge 42 and, when the tibial component 30 is mounted, towards the working surface 39 of the insert 32. Furthermore, advantageously, each of the projections 52 shows a sharp edge 55, which sharp edge 55 is preferably placed on the distal end of each projection 52, so as to effectively interfere with the bone (FIGS. 8a and 10a).

In the shown embodiments, each projection 52 is delimited by said inclined surface 54 and by an upper surface 56 perpendicular to the peripheral side surface 53 and substantially parallel to the first and the second faces 35, 36 of the base wall 34. The inclined surface 54 and the upper surface 56 converge in a common edge defining the sharp rim 55 of the distal end of the projection 52.

Furthermore, each projection 52 is delimited by a couple of hollow side surfaces 57, which connect the inclined surface 54 with the upper surface 56 in correspondence with respective edges 58 which are developed according to curved lines.

Preferably, the projections 52 are consecutively aligned along the peripheral development of the peripheral side surface 53, or on the outer surface of the side wall, so as to define a relief-border. According to what shown, the hollow side surfaces 57 of two adjacent projections 52 are uninterruptedly jointed.

In the preferred and shown embodiment, to a first series 59 of projections 52 consecutively arranged along the peripheral development of the peripheral side surface 53, a second series 60 of said projections 52, also these latter consecutively arranged along the peripheral development of the peripheral side surface 53, is flanked. The second series 60 is flanked to the first series 59 along the axial direction “Z” parallel to a generatrix of the peripheral surface 53, so as to define two relief-borders (FIGS. 8 and 9).

Furthermore, advantageously, the projections 52 of the first series 59 and the projections 52 of the second series 60 are mutually staggered.

More particularly, each projection 52 of the first series 59, relative to said axial direction “Z” parallel to a generatrix of the peripheral surface 53 and passing through the projection itself 52, is not aligned with respect to each of the projections 52 of the second series 60.

In other words, the axial direction “Z” parallel to a generatrix of the peripheral surface 53 and passing through a point of a projection 52 of the first series 59 does not pass through the point corresponding with a projections 52 of the second series 60.

In particular, in the shown embodiment, each sharp rim 55 of the distal end of a projection 52 of the first series 59 is faced to the hollow side surfaces 57 of two adjacent projections 52 of the second series 60.

According to further embodiments, not shown but falling within the ambit of the present invention, additional series of projections 52 can be present, preferably staggered therebetween, according to what above specified.

The tibial component 30 further includes a lug 61 which is developed from the first face 35 of the base wall 34 of the anchor plate 31 opposite to the seat 33 and is equipped with at least a recess 62.

The lug 61 is defined by an auxiliary wall 63 which is developed perpendicularly from the first face 35 and delimits said recess 62.

According to what shown, the auxiliary wall 63 shows a tubular shape with a circular sections which remains open in correspondence with the opposite end to the one connecting with the base wall 34 (FIG. 10).

The auxiliary wall 63 further shows, advantageously, a plurality of through-openings 64 in communication with the recess 62. Such openings 64 are defined by a plurality of holes circumferentially distributed in the auxiliary wall 63.

Advantageously, the lug 61 at least partly hollow and equipped with holes 64, allows the housing of osteoconductive material, such as the hydroxylapatite, suitable for promoting the migration of the cells responsible for the osteo-integration, and the passage of body fluids and medullar blood intended for forming the new bone material around to and within the tibial component 30.

The lug 61 can further be carried out like the pin 1 for the anchor of bone prostheses, above described and shown in the FIGS. 1 to 7.

Furthermore, advantageously, the first surface 35 of the anchor plate 31 opposite to the seat 33 further shows a plurality of through-holes 65 which are faced in the seat itself 33. In other words, the base wall 34 is perforated, so as to allow the formation of the new bone material also within the anchor plate 31. Some of such holes 65 are opened inside the recess 62 of the lug 61 (FIGS. 9 and 10).

The projections 52 and, preferably, also the lug 61 are obtained as one-piece, for example by molding and/or removal of material from a single metal block, with the anchor plate 31.

According to an embodiment shown in FIGS. 12 to 15, the lug 61 of the anchor plate 31 is decentralized with respect to the first face 35 of the base wall 34 and preferably shows an own axis with a longitudinal development “K” which delimits, relative to said first face 35, an angle “α” other than 90°. Preferably, such angle “α” is lower than 90°, preferably between 45° and 70°. More preferably, the angle “α” is equal to 60°.

Alternatively, such angle “α” is between 45° and 90° and lower than 90°, preferably such angle “α” is between 70° and 90° and lower than 90°.

Also the lug 61 of the second embodiment is defined by an auxiliary wall 63 which is developed inclined from the first face 35 and delimits the recess 62.

According to what shown, the auxiliary wall 63 presents a tubular shape with a circular section with open opposite ends 66, 67. As it is clearly shown in FIG. 7, the lug 61 is not perforated but is open both in correspondence with the proximal end 66, connected with the base wall 34, and in correspondence with the terminal end 67, opposite to the one connected to the base wall 34.

Furthermore, a first curved portion 68 of the rim of the terminal end 67 of the lug 61 is orthogonal to said axis with a longitudinal development “K”, while a second curved portion 69 of the same rim is parallel to the first face 35. The first and the second portions 68, 69 converge in two convex edges 70 which, preferably, are developed orthogonal to the direction of prevalent development “W” of the anchor plate 31 (FIG. 15).

The lug 61 further includes projections 71 arranged on an own outer side surface 72, whose projections 71, preferably show a sharp rim 73.

In the embodiment shown, each of said projections 71 and the respective rim 73 show a curved form and are developed at least partly around the axis of longitudinal development “K”.

The sharp rim 73 is substantially parallel to the first face 35 of the base wall 34.

Likewise the projections 52 arranged on the side wall 37, also the projections 71 of the lug 61 allow the instantaneous blocking of the tibial component 30 in the housing obtained within the bone tibial plate and the primary fixing of such tibial component 30 on such tibial plate.

The projections 71 further show such a structure to allow an easy introduction in the bone and to simultaneously ensure the immediate blocking.

For this purpose, preferably, each projection 71 shows a surface 74 inclined by mutual consent to the inclination of the inclined surface 54 of the projections 52 arranged on the side wall 37.

In other words, the inclined surface 74 moves away from the outer side surface 72 of the lug 61 starting from an own proximal end closer to the terminal end 67 of the lug 61 towards an own distal end closer to the proximal end 66 of the lug 61.

In the shown embodiment variation, the projections 71 are obtained by making some recess in the auxiliary wall 63, for example by molding and/or material removal.

Furthermore, preferably, as it is visible in FIG. 14, the recess 62 obtained in the lug 61 does not show a constant section along the longitudinal development of the lug 61, but it is slightly tapered towards the terminal end 67.

Preferably, the embodiment shown in FIGS. 12 to 15, is further equipped with a pin 75 (shown in FIGS. 16, 17 and 18) which is inserted in the recess 62 of the lug 61 and protrudes beyond the terminal end 67 of the lug 61 itself.

The pin 75 is preferably similar to the pin 1 for the anchor of bone prostheses above described and shown in FIGS. 1 to 7.

More particularly, the pin 75 is extending along an own rectilinear longitudinal axis “L” and includes a proximal portion 76, intended for being inserted and blocked in the recess 62 of the lug 61, and a distal portion 77 suitable for being inserted in a hole obtained in the bone.

When the pin 75 is mounted on the anchor plate 31, the distal portion 77 protrudes beyond the terminal end 67 of the lug 61 and the longitudinal axis “L” of the pin 75 corresponds with the axis of the longitudinal development “K” of the lug 61.

In the preferred and shown embodiment, both the proximal portion 76 and the distal one 77 of the pin 75 show a circular cross section.

The proximal portion 76 is frustoconical and is preferably housed and blocked by a socket in the recess 62.

A terminal edge 78 of the proximal portion 76 is shaped so as to not protruding from the first face 35 of the base wall 34 once the pin 75 is mounted on the anchor plate 31.

For this purpose, the above terminal rim 78 shows a first curved portion 79 and a second curved portion 80 which converge in two convex edges 81, likewise the shape of the terminal end 67 of the lug 61 shown in FIGS. 6, 7 and 8. The edges 81 delimit an angle of about 120°. Advantageously, the distal portion 77 of the pin 75 is at least partly deformable in a radial direction and in a resilient way, in order to allow the instantaneous blocking of the pin 75 in the hole obtained in the bone and the primary fixing of the tibial component 30 on the bone itself.

The distal portion 77 preferably includes a plurality of resilient elements 82 which are developed away from an own outer side surface 83 and which, when subjected to a compression, are movable along a radial direction with respect to the axis “L” of the pin 75, between a resting configuration and a working configuration. In the resting configuration, free ends of the resilient elements 82 are spaced from the side surface 83 of the pin 75. When the pin 75 is introduced in the hole, the internal wall of the same hole causes the deformation of the resilient elements 82 towards the axis “L” of the pin 75. Once the distal portion 77 has been inserted, the elasticity of the mentioned elements 82 pushes the free ends of the same against the internal wall of the hole, by holding the pin 75 in the bone.

More particularly, according to what shown in the appended figures, the resilient elements 82 of the distal portion 77 are defined by a plurality of resilient wings which are developed from the outer surface 83.

Preferably, each wing 82 is joined to the distal portion 77 in correspondence with an own restrained end opposite to the free end, and mostly extending along a direction parallel to the longitudinal axis “L” of the pin 75.

Moreover, advantageously, the distal portion 77 is at least partly hollow and equipped with openings 84, so as to allow the housing of osteo-inductive material, as the hydroxylapatite, and the passage of body fluids and medullar blood intended for forming the new bone material around to and within the pin 75.

In the preferred embodiment shown in FIGS. 16, 17 and 18, each wing 82 shows a triangular shape with a side 85 lying on a directrix circumference of the cylindrical side surface 83 and joined to such surface 83 and a free vertex faced towards the proximal portion 76.

Each wing 82 is obtained through a couple of rectilinear notches which are joined up in a crossing point and define a triangular main opening 86.

The pin 75 of the shown embodiment shows three series of wings 82, each series is axially and angularly staggered by the adjacent series and includes three angularly equispaced wings 82 and the same number of circular auxiliary openings 84.

The pin 75 is hollow and an end 87 of the distal portion 77 is open, so as to allow the housing of a small cylinder of the said osteoinductive mineral material, suitable for promoting the migration of the cells responsible for the osteo-integration.

In use, after a proper housing has been obtained in the tibial plate of the patient, the anchor plate 31 is forced in such housing such that the terminal rim 42 of the side wall 37 substantially remains flush with the bone surface and the projections 52, 73 enter into contact and interfere with internal walls of the above housing.

The lug 61 and the pin 75, if necessary, are inserted in a suitable hole obtained on the bottom of the above housing.

The insert 32 remains outside the housing and the working surface 39 faced to and contacting the plate 3 of the condyle-femoral component, for example as the one diagrammatically shown in FIG. 7.

The tibial component 30 according to the invention attains important advantages. In fact, the presence of the projections allows an easy primary fixing of the prosthesis on the bone, by simply pressure fitting the plate in the tibial plate.

The conformation of the projections allows the introduction of the tibial component but it safely prevents any movement thereof when the installation has been carried out.

Such advantage means a remarkable simplification of the surgical procedure, as it does not need the positioning in the bone housing of multiple elements, such as the screws, which represent the traditional fixing system, nor the use of cement for the temporary blocking.

Furthermore, the insert is easily joined to the anchor plate before the implant in the bone of the patient through a snap-coupling, making this operation quite fast and safe.

The hollow structure of the lug and the pin and the presence of openings in the base wall and in the pin allow the migration of cell components, sproutings of autologous bone and nutriment fluids rich of growth factors within the cylindrical empty structure of the lug. All these biological components represent a determining factor for the bone neoplasia process.

Finally, the filling of the empty space of the lug of the anchor plate or the pin with a small cylinder of biomimetic bone substitute based on hydroxylapatite with a high porosity, simply placed by a “press-fit”, or alternatively a bone substitute in form of an injectable paste, represents a factor promoting the bone neoplasia process and accelerates, de facto, the osteo-integration within the seat obtained in the tibial bone. The scaffold will be occupied by a cell population which will undergo, thanks to the biomimetic properties of the same, a differentiation in an osteoblastic sense with a consequent synthesis of bone matrix and promoting, thanks to it micro-porous network, the new angiogenesis.

The knee prosthesis, generally formed by the condyle-femoral component 2 (exemplified in FIG. 7) and the tibial component 30 (exemplified in FIG. 8), includes a pin 1 associated with the plate 3 of said condyle-femoral component 2 as above described and/or claimed in one or more of the claims 1 to 11. Furthermore, or alternatively to the pin 1 as above described and/or claimed in one or more of the claims 1 to 11, the mentioned prosthesis generally presents the features of the tibial component 30 as described and/or claimed in one or more of the claims 21 to 55. In other words, the knee prosthesis can generally show the pin 1 according to the invention for the fixing of the condyle-femoral component 2 and a traditional tibial component or a traditional pin for the fixing of the condyle-femoral component 2 and the tibial component 30 according to the invention or the pin 1 according to the invention and the tibial component 30 according to the invention.

1-57. (canceled)

58. Anchor pin of articular prostheses including a connecting portion with a prosthesis and an engagement portion in a hole obtained in a bone; wherein the engagement portion is at least partly deformable in a radial direction and in a resilient way, in order to allow the instantaneous blocking of the pin in the hole and the primary fixing of said prosthesis on said bone;
wherein the engagement portion shows a plurality of resilient wings developing away from an outer surface of said engagement portion and intended for interfering with an internal wall of the hole obtained in the bone;
wherein the engagement portion is at least partly hollow and equipped with openings for allowing the housing of osteoinductive material and the passage of medullar fluids until the formation of bone bridges;
wherein the engagement portion is defined by a cylindrical wall showing a plurality of notches delimiting the wings and defining said openings.

59. Pin according to claim 58, wherein each of said wings shows a free end faced towards the connecting portion.

60. Pin according to claim 59, wherein the free end of the wings is pointed.

61. Pin according to claim 58, wherein each wing shows a triangular shape with a side joined to the cylindrical wall and a free vertex faced towards the connecting portion.

62. Pin according to claim 58, wherein each wing shows a rectangular shape mainly developing along a direction parallel to a longitudinal axis (X) of the pin, with a shorter side joined to the cylindrical wall and a free end faced towards the connecting portion.

63. Pin according to claim 61, wherein each wing is curved and shows a concavity faced towards the outside of the pin.

64. Pin according to claim 62, wherein each wing is curved and shows a concavity faced towards the outside of the pin.

65. Pin according to claim 58, wherein an end of the engagement portion opposite to the connecting portion is open, in order to allow the housing of a small cylinder of said osteoinductive material.

66. Pin according to claim 58, wherein the connecting portion shows a threading for the engagement with the prosthesis.

67. Articular prosthesis including at least a pin according to claim 58.

68. Prosthesis according to claim 67, characterized in that it is a knee prosthesis.

69. Prosthesis according to claim 67, characterized in that it is an articular prosthesis of the shoulder.

70. Prosthesis according to claim 67, characterized in that it is an articular prosthesis of the ankle.

71. Prosthesis according to claim 67, characterized in that it is a femoral half-moon.

72. Prosthesis according to claim 67, characterized in that it is a tibial plate.

73. Prosthesis according to claim 67, wherein the pin is screwed on the prosthesis.

74. Prosthesis according to claim 67, wherein the pin is welded on the prosthesis.

75. Prosthesis according to claim 67, wherein the pin is integrally carried out with the prosthesis.

76. Knee articular prosthesis, including a half-moon shaped plate and intended for being associated with a femoral condyle; at least one pin according to claim 58 and associated with the plate; a tibial component including an anchor plate which can be connected to a bone tibial plate and an insert which can be mounted in a seat obtained in the anchor plate; the anchor plate including a plurality of projections arranged on an own peripheral side surface, in order to allow the instantaneous blocking of the tibial component in a housing obtained in the bone tibial plate and the primary fixing of said tibial component on said tibial plate.
